(19) United States
(12) Patent Application Publication
(10) Pub. No.: US 2026/0053139 A1
(43) Pub. Date: Feb. 26, 2026

(54) SHORT PEPTIDE NP19, POLYPEPTIDE Nlsp5 AND APPLICATION THEREOF

(71) Applicant: Jiangsu Academy of Agricultural Sciences, Nanjing (CN)

(72) Inventors: Rui Ji, Nanjing (CN); Liangxuan Qi, Nanjing (CN); Jizhao Fang, Nanjing (CN); Jing Li, Nanjing (CN); Han Wang, Nanjing (CN); Jing Li, Nanjing (CN); Guanghua Luo, Nanjing (CN)

(21) Appl. No.: 19/385,403

(22) Filed: Nov. 11, 2025

(30) Foreign Application Priority Data
Mar. 27, 2025 (CN) 202510369638.0

(51) Int. Cl. A01N 37/46; A01P 7/04; C07K 14/435

(52) U.S. Cl. CPC A01N 37/46; A01P 7/04; C07K 14/43563

(57) ABSTRACT

Provided in the present disclosure are a short peptide NP19, a polypeptide Nlsp5 and application thereof, which belong to the field of bioengineering technology. The short peptide NP19 and the polypeptide Nlsp5 can significantly activate the plant immune system, improve the insect resistance of plants, enhance the ability of plants to resist insects, and effectively prevent or reduce the occurrence of insect pests. The short peptide NP19 and the polypeptide Nlsp5 of the present disclosure provide a new way to improve the insect resistance of plants, provide resources for the future development and application of biopesticides, can be used as a new type of microbial protein pesticide to defend against insect pests, and also provide a basis for the future transgenic plants of this gene, thus having broad application prospects in agricultural production.

### SEQUENCE LISTING

The sequence listing is submitted as an XML file filed via EFS-Web, with a file name of “Sequence_Listing.XML”, a creation date of Nov. 11, 2025, and a size of 18,927 bytes. The sequence Listing filed via EFS-Web is a part of the specification and is incorporated in its entirety by reference herein.

### TECHNICAL FIELD

The present disclosure relates to a short peptide NP19, a polypeptide Nlsp5, and application thereof, belonging to the field of bioengineering technology.

### BACKGROUND

In recent years, biological control methods have been increasingly widely used in pest control. Biological control utilizes natural enemies or microorganisms in nature to control pests. For example, natural enemies such as parasitic wasps, predatory insects, fungi, bacteria, etc. are used to suppress the population of pests. In addition, the inherent insect-resistant ability of plants has attracted increasing attention. Through studies on the natural insect-resistant mechanisms of plants, scientists have found that many plants can resist pests by secreting specific chemical substances or activating immune responses. When attacked by pests, these plants can respond rapidly by activating their immune systems and releasing some natural insect-resistant elicitors, thereby suppressing the growth and reproduction of pests.

However, current biological control technologies face some challenges. First, the effectiveness of biological control is usually greatly affected by environmental conditions. Some natural enemies or microorganisms may not perform optimally under different climatic and soil conditions. Second, although some natural insect-resistant elicitors have been identified, the mechanisms of these substances are not fully understood, and the stability and persistence of their application are still difficulties in research. Therefore, the screening of highly efficient and stable insect-resistant elicitors has become an important research direction.

In recent years, the screening and identification of insect-resistant elicitors has become a research hotspot in the field of biological control. Through in-depth studies on plant immune responses, scientists have gradually revealed the roles of various natural insect-resistant elicitors, such as certain proteins, carbohydrates, and small molecule compounds, in the insect-resistant mechanism. In particular, the immune signaling pathways mediated by pattern recognition receptors (PRRs) have been demonstrated to play an important role in plants' defense against pests. Based on these findings, researchers are exploring how to improve the insect-resistant ability of plants through exogenous application of these elicitors or through genetic engineering means.

However, despite the discovery and preliminary application of some insect-resistant elicitors, there are still many challenges in how to improve their application effects in practical agricultural production. For example, problems such as how to improve the absorption efficiency of elicitors in plants, prolong their action time in plants, enhance their broad-spectrum property and stability against different types of pests, etc. are still key technical problems that need to be solved urgently.

Therefore, the development of novel insect-resistant elicitors or the improvement of the plants' own immune systems through genetic engineering to enhance their defense ability against pests is an important direction for the current development of biological control technologies.

### SUMMARY

To solve the above-mentioned defects and deficiencies existing in the prior art, the present disclosure provides a short peptide NP19, a polypeptide Nlsp5 and application thereof, which can effectively improve the insect resistance of tobacco or rice.

To solve the above-mentioned technical problems:

The first object of the present disclosure is to provide a short peptide NP19 derived from the salivary sheath secretory protein of Nilaparvata lugens (Stal), and the amino acid sequence of the short peptide NP19 is as set forth in SEQ ID NO: 3.

Furthermore, the nucleotide sequence encoding the short peptide NP19 is as set forth in SEQ ID NO: 4.

The second object of the present disclosure is to provide a salivary sheath secretory polypeptide Nlsp5 of Nilaparvata lugens (Stal), including the above-mentioned short peptide NP19.

Furthermore, the amino acid sequence of the polypeptide Nlsp5 is as set forth in SEQ ID NO: 1.

Furthermore, the nucleotide sequence encoding the polypeptide Nlsp5 is as set forth in SEQ ID NO: 2.

The third object of the present disclosure is to provide application of the short peptide NP19 or the polypeptide Nlsp5 in improving the insect resistance of tobacco or rice.

Furthermore, the short peptide NP19 or the polypeptide Nlsp5 is used as an elicitor to induce defense responses or hypersensitive responses in tobacco or rice.

Furthermore, the short peptide NP19 or the polypeptide Nlsp5 is sprayed on tobacco or rice as a pesticide or as a main component of a pesticide.

The fourth object of the present disclosure is to provide application of the short peptide NP19 or the polypeptide Nlsp5 in the preparation of a pesticide.

The beneficial technical effects achieved by the present disclosure: The short peptide NP19, the polypeptide Nlsp5 and application thereof provided by the present disclosure can significantly activate the plant immune system, improve the insect resistance of plants, enhance the ability of plants to resist insects, and effectively prevent or reduce the occurrence of insect pests. The short peptide NP19 and the polypeptide Nlsp5 of the present disclosure provide a new way to improve the insect resistance of plants, provide resources for the future development and application of biopesticides, can be used as a new type of microbial protein pesticide to defend against insect pests, and also provide a basis for the future transgenic plants of this gene, thus having broad application prospects in agricultural production.

### BRIEF DESCRIPTION OF THE DRAWINGS

FIG. 1 shows the results of Nlsp5-induced cell death and reactive oxygen species (ROS) production in Nicotiana benthamiana;

FIG. 2 shows the results of expression level of Nlsp5 in transgenic rice;

FIG. 3 shows the results of Nlsp5-induced defense factor in rice;

FIG. 4 shows the results of Nlsp5-induced resistance of rice against Nilaparvata lugens (Stal);

FIG. 5 shows the results of Nlsp5-induced resistance of rice against Laodelphax striatellus (Fallen);

FIG. 6 shows the results of Nlsp5-induced field resistance of rice against three planthopper species;

FIG. 7 shows the results of Nlsp5-induced resistance of rice against Chilo suppressalis;

FIG. 8 shows the results of NP19-induced cell death and reactive oxygen species (ROS) production in Nicotiana benthamiana;

FIG. 9 shows the results of NP19-induced expression of defense-related genes in Nicotiana benthamiana;

FIG. 10 shows the results of NP19-induced defense factor in rice;

FIG. 11 shows the results of NP19-induced resistance of Nicotiana benthamiana against Bemisia tabaci;

FIG. 12 shows the results of NP19-induced resistance of rice against Nilaparvata lugens (Stal) and Laodelphax striatellus (Fallen);

FIG. 13 shows the results of NP19-induced resistance of rice against Chilo suppressalis;

FIG. 14 shows the results of NP19-induced cell death in different plant species;

FIG. 15 shows the gel electrophoresis bands of Agrobacterium detected in the present disclosure; and

FIG. 16 shows the results of insect resistance induced by the short peptide NP19 of the present disclosure in cotton.

### DETAILED DESCRIPTION OF THE EMBODIMENTS

The present disclosure will be further described below in conjunction with specific embodiments. The following embodiments are only used to illustrate the technical solution of the present disclosure more clearly and cannot be used to limit the protection scope of the present disclosure.

The present disclosure will be further illustrated below in conjunction with the accompanying drawings and embodiments.

The polypeptide Nlsp5 and the short peptide NP19 disclosed in the present disclosure can improve the resistance of different plants against different insects. To illustrate the insect resistance of the polypeptide NIsp5 and the short peptide NP19 of the present disclosure more clearly, some plant species, such as rice, tobacco, and cotton, are selected for detailed description; however, this is not intended to limit the scope of protection of the present disclosure. Likewise, with respect to insect species, the following embodiments select Nilaparvata lugens (Stal), Laodelphax striatellus (Fallen), Sogatella furcifera (Horvith), Bemisia tabaci, Chilo suppressalis, etc. for detailed description, but this does not mean that the short peptide and polypeptide of the present disclosure have no resistance against other insects.

### Embodiment 1 Construction of Transgenic Plants Expressing Polypeptide Nlsp5

1. Preparation of target protein: The salivary sheath secretory proteins were collected from Nilaparvata lugens (Stal) for protein sequencing to obtain their nucleotide sequences. The nucleotide sequences of different proteins were respectively constructed into the transient expression vector pBINPLUS. The resulting expression vector was then transformed into Agrobacterium and injected into Nicotiana benthamiana. A protein that can induce hypersensitive cell death in Nicotiana benthamiana was subsequently screened and designated as Nlsp5, the amino acid sequence of which is as set forth in SEQ ID NO: 1, and the nucleotide sequence encoding the polypeptide Nlsp5 is as set forth in SEQ ID NO: 2.

SEQ ID NO: 1:
MASFTSLLSALVLVAGSQGVAVNPMGMADAGLGMVDAGVSAGMGLASTG
MDAGKGLANAGMGAASSMSGRMTEEAKKLNGNIQDMLGAQMKGGANILN
NMLKMDAAVLTEMGTMISSIAHLTGATSQQMLQILQEVMTKGPLAGAQM
LQRLLAYIAQKKIAAGDKIVSKTSSFADGMKQKSFIPLNMLDNMYRTLG
SVNDITSALTGTPSLAASKAATALTPPVV.
SEQ ID NO: 2:
ATGGCTTCATTCACATCTCTATTATCCGCTCTTGTGTTGGTCGCTGGTT
CACAGGGAGTGGCTGTGAACCCAATGGGAATGGCGGATGCAGGACTGGG
AATGGTGGATGCAGGAGTTTCAGCGGGAATGGGACTGGCAAGTACTGGA
ATGGATGCAGGAAAGGGCTTGGCGAATGCAGGAATGGGTGCTGCTAGTT
CCATGTCTGGTCGCATGACGGAGGAAGCTAAAAAATTAAATGGAAACAT
TCAGGACATGTTAGGAGCGCAGATGAAGGGAGGTGCAAACATACTGAAT
AATATGTTGAAGATGGACGCTGCCGTTCTCACTGAAATGGGGACAATGA
TCAGTTCTATTGCACACTTAACTGGAGCAACATCACAACAGATGTTGCA
AATACTACAAGAAGTCATGACGAAAGGACCACTCGCAGGTGCTCAAATG
TTACAGAGGTTATTAGCATACATTGCACAAAAGAAGATAGCAGCAGGTG
ATAAGATTGTTTCCAAGACCAGCAGCTTCGCTGATGGCATGAAGCAAAA
ATCGTTTATACCATTAAACATGTTGGATAATATGTACAGAACGCTTGGA
TCAGTTAACGATATAACATCAGCATTGACTGGCACTCCTTCGCTTGCCG
CATCAAAAGCAGCTACAGCCCTCACTCCACCCGTAGTTTGA.

2. Construction of plasmid: Under the control of the CaMV35S promoter, the nucleotide sequence encoding the polypeptide Nlsp5 was ligated into the vector pCAMBIA1301 to obtain plasmid pCAMBIA1301-Nlsp5.

Processes 1 and 2 were performed by Wuhan Biorun Biological Technology Co., Ltd.

3. Transformation of plasmid: 1 μl of the plasmid pCAMBIA1301-Nlsp5 was added to 50 μl of EHA105 Agrobacterium competent cells. After thorough mixing, the mixture was aspirated into an electroporation cuvette and subjected to electroporation. Subsequently, 1 ml of LB liquid medium was added, thoroughly mixed and aspirated into a 1.5 ml centrifuge tube. The mixture was incubated on a shaker at 30° C. and 180 rpm for 30 min. 50 μl of the activated Agrobacterium solution was aspirated onto LB solid medium and incubated at 30° C. in the dark for 48 h.

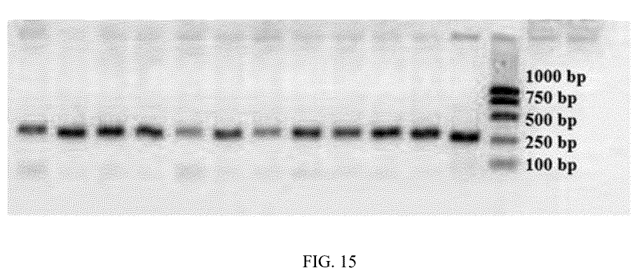

4. Detection of Agrobacterium: Corresponding detection primers were synthesized (the base sequence of the forward primer F is as set forth in SEQ ID NO:15: TACTGGAATGGATGCAGGA; and the base sequence of the reverse primer R is as set forth in SEQ ID NO:16: ACATTTGAGCACCTGCGAGT). A PCR system was prepared, and detection was performed by gel electrophoresis. A 1% agarose gel was prepared, followed by sample loading. The results were shown in FIG. 15, where clear and correctly sized electrophoretic bands were observed in the positive control and in the samples, while no band was observed in the negative control, indicating that the sample could proceed to the next step.

5. Induced Transformation and Callus Preparation

Preparation: Required experimental equipment and reagents were prepared, including rice seeds without mildew spots and with normal germ openings, an alcohol lamp, alcohol swabs (75% pure alcohol), a lighter, culture dishes, disposable rubber gloves, large forceps, small forceps, filter paper (sterilized), a timer, 4.5% sodium hypochlorite, empty Erlenmeyer flasks (sterilized), sterile water, sealing film, MS induction medium (containing 3 mg/L of 2,4-D), agar, sucrose, and a waste liquid cylinder, etc.

Sterilization of laminar flow hood: The laminar flow hood was wiped with alcohol swabs, and experimental materials were placed inside the hood and irradiated with UV light for more than half an hour, followed by air drying for more than 10 minutes.

Sterilization of seeds: The seeds were placed in an empty Erlenmeyer flask and sterilized with 75% ethanol for 1 min, followed by washing once with sterile water for 1 min. The seeds were then sterilized with sodium hypochlorite for 15 min, with shaking several times every minute. After discarding the sodium hypochlorite, the seeds were washed five times with sterile water for 2 min each time. The sterile water was discarded, and the seeds were transferred onto culture dishes containing filter paper to dry.

Inoculation: Small forceps were heated over an alcohol lamp until they turned red, and after cooling, were used to transfer the dried seeds into induction medium (1 L of NB medium containing 300 mg/L of hydrolyzed casein, 500 mg/L of glutamine, 500 mg/L of proline, 100 mg/L of inositol, 30 g/L of sucrose, 2.6 mg/L of PHytagel, and NB+2 mg/L of 2,4-D), with approximately 15 seeds per dish. The culture dishes were sealed with 2-3 layers of sealing film.

Culture: The culture dishes were placed in an incubator for culture, waiting for the formation of calli.

6. Infection of Agrobacterium: Agrobacterium was picked and transferred into an infection solution (500 μL of 1M MgCl₂, 1 mL of 0.5M MES, pH 5.7, 50 μL of 100 mM Acetosyringone, ddH₂O added to 50 mL, freshly prepared before use) to prepare an Agrobacterium resuspension with an OD₆₀₀ of 0.2. Calli were picked into an Erlenmeyer flask and infected with the Agrobacterium resuspension for 10-15 h. After then, the bacterial solution was discarded, and the calli were inoculated onto the co-culture medium (NB+2 mg/L of 2,4-D+100 μM/L of AS), and co-cultured at 20° C. for 48-72 h.

7. Screening of callus: the calli from step 6 were inoculated onto a screening medium (NB+2 mg/L of 2,4-D, 250 mg/L of Car, 30 mg/L of Hyg) and incubated at 26° C. in the dark for 20-30 days. The positive calli were inoculated onto a secondary screening medium (NB+2 mg/L of 2,4-D, 50 mg/L of Hyg). During the callus picking process, monoclonal calli should be picked and incubated at 26° C. in the dark for 7-10 days.

8. Differentiation and rooting: the positive calli were inoculated onto a differentiation medium (NB, 10 mg/L of KT, 0.4 mg/L of NAA) and incubated under light at 25-27° C. for 15-20 days. After the differentiated buds grew to 2-5 cm, the buds were inoculated onto a rooting medium (½ MS inorganic salts, MS organic components, and 30 mg/L of Hyg) and incubated under light at 30° C. for 7-10 days.

9. Detection of positive seedlings: Genomic DNA of rice was extracted using the CTAB method. Homozygous transgenic plants were screened by hygromycin resistance, GUS staining, and PCR. The expression of Nlsp5 gene in rice was verified by qRT-PCR. The primers for PCR screening of the strains are as follows:

Nlsp5-oe-F:
5′-GTTAGGAGCGCAGATGAAGG-3′,
as set forth in SEQ ID NO: 5;
Nlsp5-oe-R:
5′-ACATTTGAGCACCTGCGAGT-3′,
as set forth in SEQ ID NO: 6;
the primers for verifying the expression of the
Nlsp5 gene in rice by qRT-PCR are as follows:
RT-Nlsp5-oe-F:
5′-AGATTGTTTCCAAGACCAGCA-3′,
as set forth in SEQ ID NO: 7;
RT-Nlsp5-oe-R:
5′-CGTTAACTGATCCAAGCGTTC-3′,
as set forth in SEQ ID NO: 8.

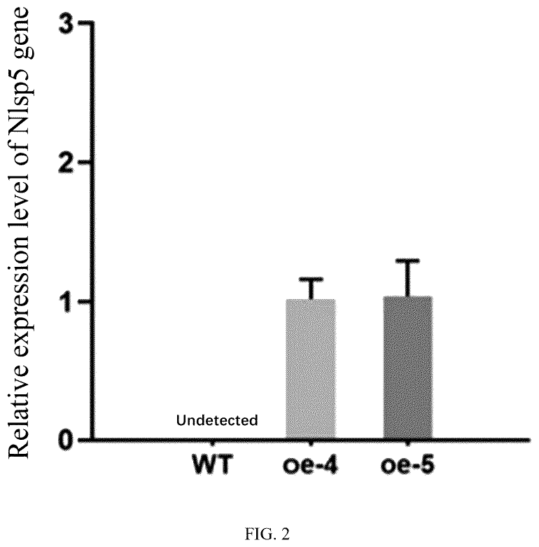

Results: As verified by qRT-PCR, the expression of the Nlsp5 gene was not detected in wild-type rice (WT), while the expression of the Nlsp5 gene was detected in both homozygous transgenic plants oe4 and oe5, as shown in FIG. 2.

### Embodiment 2 Induction of Defense Factors in Transgenic Rice by the Polypeptide Nlsp5

Transgenic rice expressing Nlsp5 from Embodiment 1 and wild-type rice were selected, and rice stems were placed into a glass cylinder containing 20 newly hatched female adults of Nilaparvata lugens (Stal). After 8 and 24 hours, the Nilaparvata lugens (Stal) were removed, and the rice stems were collected, respectively. The samples were ground in liquid nitrogen, extracted with ethyl acetate containing labeled internal standards (²D₄-SA, ²D₆-JA, and ²D₆-JA-Ile), and analyzed for salicylic acid, jasmonic acid, and jasmonic acid-isoleucine using high-performance liquid chromatography-tandem mass spectrometry. Hydrogen peroxide concentrations were quantitatively analyzed using the AmplexRed hydrogen peroxide/peroxidase assay kit (Invitrogen).

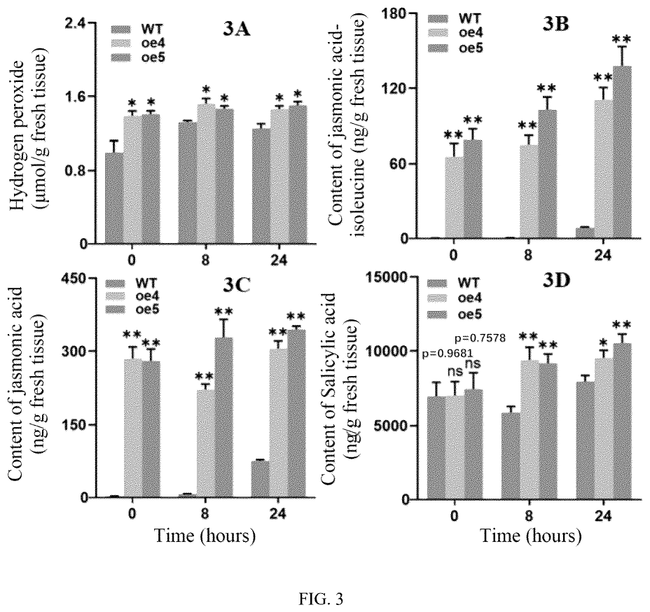

Results: The results of quantitative analysis of hormone concentrations showed that the introduction of the Nlsp5 gene into rice could significantly induce increase in the contents of hydrogen peroxide, jasmonic acid and jasmonic acid-isoleucine, as shown in FIG. 3. Among them, 3A shows the content of hydrogen peroxide, 3B shows the content of jasmonic acid-isoleucine, 3C shows the content of jasmonic acid, and 3D shows the content of salicylic acid.

### Embodiment 3 Induction of Defense Factors in Tobacco by the Polypeptide Nlsp5

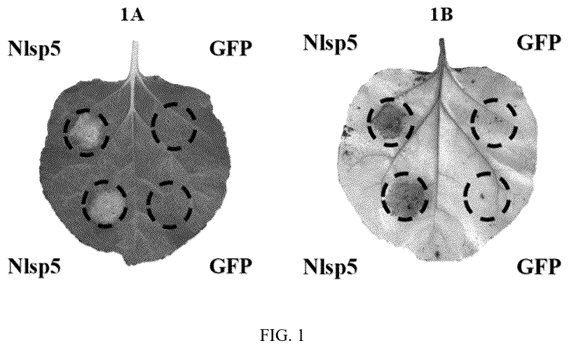

The constructed pBINPLUS-Nlsp5 vector was introduced into Agrobacterium tumefaciens GV3101 strains using the electroporation method. The Agrobacterium GV3101 strains were incubated in an LB liquid medium containing kanamycin and rifampicin at 28° C. for 24 hours. The recombinant strains were washed three times with an infiltration buffer (1 M MgCl₂, 100 mM MES, 150 mM acetosyringone) and resuspended to an OD₆₀₀ of 0.4. The Agrobacterium tumefaciens cell suspension was injected into the leaves of Nicotiana benthamiana using a needleless syringe. The results are shown in FIG. 1: the polypeptide Nlsp5 can induce visible hypersensitive cell death in Nicotiana benthamiana, where 1A shows cell death and 1B shows ROS burst.

### Embodiment 4 Induction of Insect Resistance in Transgenic Rice by the Polypeptide Nlsp5

(1) Induction of Resistance of Rice Against Nilaparvata lugens (Stal)

The Nlsp5 transgenic rice plants (oe4 strains or oe5 strains) obtained by the method of Embodiment 1 and wild-type rice plants were enclosed in a glass cylinder (with a diameter of 8 cm and a height of 8 cm), and 15 fourth-instar nymphs were released. The number of Nilaparvata lugens (Stal) nymphs on each plant was counted at 1, 2, 4, 8, 24, and 48 hours after release. For the fecundity testing, one female Nilaparvata lugens (Stal) and two male Nilaparvata lugens (Stal) were placed on the rice stems (0-8 cm above the ground) enclosed in a glass cylinder. Seven days later, the number of eggs laid by each female was counted under an optical microscope.

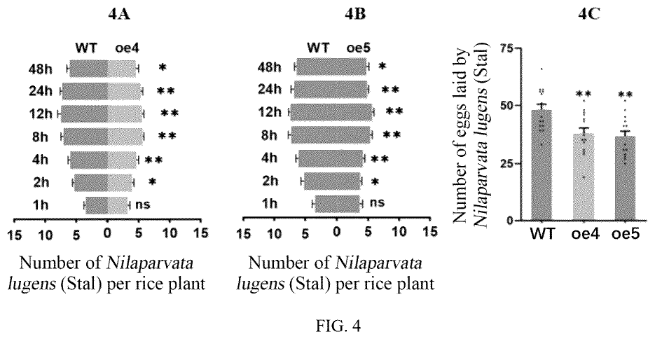

Results: After the Nlsp5 gene was introduced into rice plants, the number of Nilaparvata lugens (Stal) residing on the rice plants could be significantly reduced, and the egg-laying amount of Nilaparvata lugens (Stal) could be significantly decreased, indicating that Nlsp5 can induce the resistance of rice against Nilaparvata lugens (Stal), as shown in FIG. 4. Among them, 4A shows the number of Nilaparvata lugens (Stal) on each rice plant of oe4 strain, 4B shows the number of Nilaparvata lugens (Stal) on each rice plant of oe5 strain, and 4C shows the number of eggs laid by Nilaparvata lugens (Stal) on different plants.

(2) Induction of Resistance of Rice Against Laodelphax striatellus (Fallen)

The Nlsp5 transgenic rice plants (oe4 strains or oe5 strains) obtained by the method of Embodiment 1 and wild-type rice plants were enclosed in a glass cylinder (with a diameter of 8 cm and a height of 8 cm), and 15 fourth-instar nymphs were released. The number of Laodelphax striatellus (Fallen) nymphs on each plant was counted at 1, 2, 4, 8, 24, and 48 hours after release. For the fecundity testing, one female Laodelphax striatellus (Fallen) and two male Laodelphax striatellus (Fallen) were placed on the rice stems (0-8 cm above the ground) enclosed in a glass cylinder. Seven days later, the number of eggs laid by each female was counted under an optical microscope.

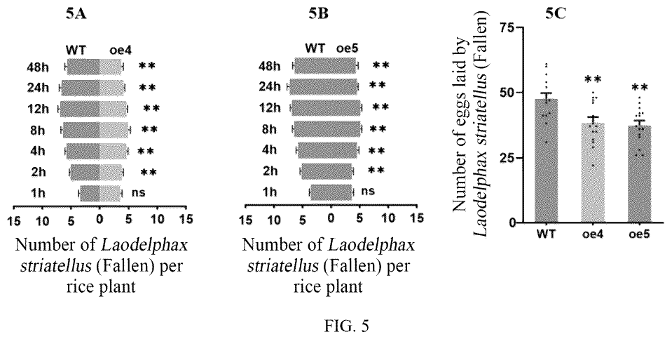

Results: After the Nlsp5 gene was introduced into rice plants, the number of Laodelphax striatellus (Fallen) residing on the rice plants could be significantly reduced, and the egg-laying amount of Laodelphax striatellus (Fallen) could be significantly decreased, indicating that Nlsp5 can induce the resistance of rice against Laodelphax striatellus (Fallen), as shown in FIG. 5. Among them, 5A shows the number of Laodelphax striatellus (Fallen) on each rice plant of oe4 strain, 5B shows the number of Laodelphax striatellus (Fallen) on each rice plant of oe5 strain, and 5C shows the number of eggs laid by Laodelphax striatellus (Fallen) on different plants.

### (3) Induction of Field Resistance of Rice Against Three Planthopper Species

Twenty-four Nlsp5 transgenic rice plants (oe4 strains or oe5 strains) obtained by the method of Embodiment 1 and twenty-four wild-type rice plants were planted in the field and covered with a net with holes. Approximately 500 Nilaparvata lugens (Stal), 500 Laodelphax striatellus (Fallen), and 500 Sogatella furcifera (Horvith) were released into the field. At 40, 54, and 68 days after planting the rice, the total number of all planthoppers (including both nymphs and adults) on each rice plant was counted.

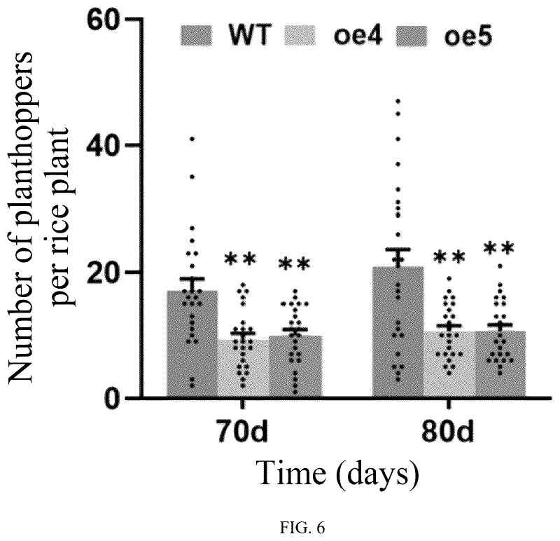

Results: After the Nlsp5 gene was introduced into rice plants, the number of the three planthopper species residing on the rice plants could be significantly reduced, indicating that NIsp5 can induce the field resistance of rice against the three planthopper species, as shown in FIG. 6.

(4) Induction of Resistance of Rice Against Chilo suppressalis

The Nlsp5 transgenic rice plants (oe4 strains or oe5 strains) obtained by the method of Embodiment 1 and wild-type rice plants were enclosed in a glass cylinder (with a diameter of 8 cm and a height of 8 cm). After weighing, 5 second-instar Chilo suppressalis nymphs were placed into the glass cylinder and attached to the basal stems. After 7 days of feeding, each insect was reweighed to evaluate the weight gain.

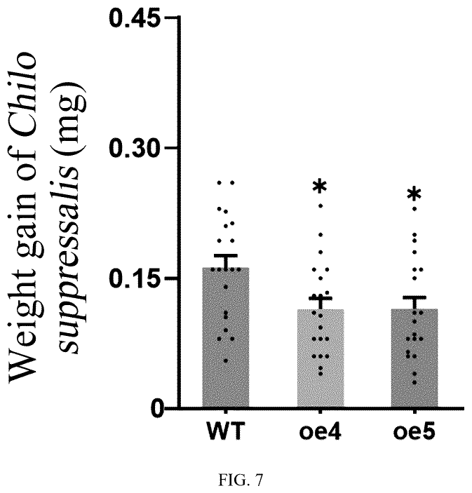

Results: After the Nlsp5 gene was introduced into rice plants, the feeding of Chilo suppressalis could be significantly reduced, indicating that Nlsp5 can induce the resistance of rice against Chilo suppressalis, as shown in FIG. 7.

### Embodiment 5 Induction of Defense Responses of Plants by the Short Peptide NP19

1. Synthesis of short peptide NP19: In the laboratory, it was revealed through sequence alignment that the polypeptide Nlsp5 sequence contains a conserved domain. A short peptide NP19 was obtained by truncating this protein. The amino acid sequence of the short peptide NP19 is as set forth in SEQ ID NO:3 in the Sequence Listing, and the nucleotide sequence encoding the short peptide NP19 is as set forth in SEQ ID NO:4 in the Sequence Listing. The nucleotide sequence of the short peptide NP19 was synthesized by GenScript.

SEQ ID NO: 3:
KGPLAGAQMLQRLLAYIAQ;
SEQ ID NO: 4:
AAAGGACCACTCGCAGGTGCTCAAATGTTACAGAGGTTATTAGCATACA
TTGCACAA.

2. Induction of cell death and reactive oxygen species (ROS) production in Nicotiana benthamiana leaves: The concentration of NP19 was adjusted to 1 nM, 20 nM, 50 nM, 100 nM, 500 nM, and 1 μM. Approximately 4-week-old Nicotiana benthamiana plants were selected, and the NP19 short peptide was injected into the Nicotiana benthamiana leaves from the back of the leaves using a 1-ml needleless syringe, with pure water serving as a control at the same time. After 48 hours of injection, the hypersensitive necrosis responses were observed. After 12 hours of injection, the treated leaves were placed into DAB staining solution (1 mg/ml, pH=3.8) and treated in the dark at room temperature for 8 hours. Then, the staining solution was removed, and absolute ethanol was added for decolorization. After all the green color of the leaves disappeared, the leaves were taken out and photographed.

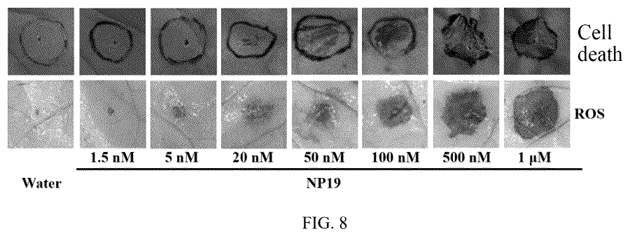

Results: NP19 at a concentration of 50 nM or higher can induce visible hypersensitive cell death on Nicotiana benthamiana. After the leaves were treated with NP19 for 12 hours, obvious brown deposits appeared at the injection site. Moreover, as the concentration of the injected short peptide increased, the area of the brown deposits became larger and the color became darker, indicating that NP19 induced ROS production in the Nicotiana benthamiana leaves, as shown in FIG. 8.

3. Induction of defense-related gene expression in Nicotiana benthamiana: After injecting the NP19 short peptide and pure water into Nicotiana benthamiana leaves, samples are taken at 24 h and 48 h, respectively. RNA was extracted using a plant RNA extraction kit, and genomic DNA was removed to obtain high-purity RNA. First-strand cDNA was synthesized using a reverse transcription kit. According to the instructions of a quantitative kit, 2 μL of the reverse transcription product was used as the template and real-time fluorescence quantitative PCR was performed using EF-la as the internal reference gene to measure the expression levels of jasmonic acid signal-related genes NbPR3 and NbPR4, which are related to the resistance of Nicotiana benthamiana. The primers used are as follows:

NbEF1a-QF:
5′-AGAGGCCCTCAGACAAAC-3′,
as set forth in SEQ ID NO: 9;
NbEF1a-QR:
5′-TAGGTCCAAAGGTCACAA-3′,
as set forth in SEQ ID NO: 10;
NbPR3-QF:
5′-TGGGGTTATTGCTGGCTTAG-3′,
as set forth in SEQ ID NO: 11;
NbPR3-QR:
5′-GGGTCATCCAAAACCAGAGA-3′,
as set forth in SEQ ID NO: 12;
NbPR4-QF:
5′-GGCCAAGATTCCTGTGGTAGAT-3′,
as set forth in SEQ ID NO: 13;
NbPR4-QR:
5′-CACTGTTGTTTGAGTTCCTGTTCCT-3′,
as set forth in SEQ ID NO: 14.

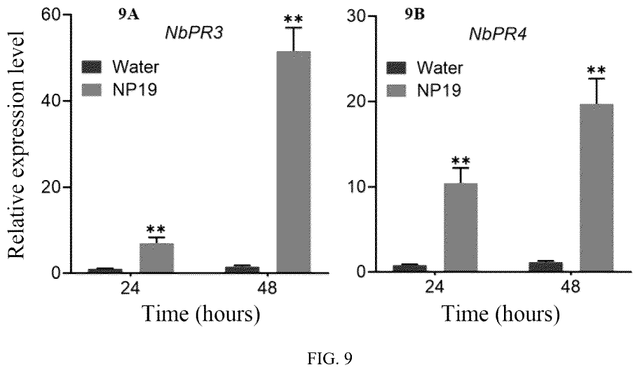

Results: The results of fluorescence quantitative PCR showed that the NP19 short peptide could significantly induce the expression of jasmonic acid signal-related genes NbPR3 and NbPR4 in Nicotiana benthamiana 1 and 2 days after injection, as shown in FIG. 9. Among them, 9A shows the expression level of the related gene NbPR3, and 9B shows the expression level of the related gene NbPR4.

4. Induction of defense factors in rice: After treating rice stems with the NP19 short peptide and water, the rice stems were collected, the samples were ground in liquid nitrogen, extracted with ethyl acetate containing labeled internal standards (²D₄-SA, ²D₆-JA, and ²D₆-JA-Ile), and analyzed for salicylic acid, jasmonic acid, and jasmonic acid-isoleucine using high-performance liquid chromatography-tandem mass spectrometry. Hydrogen peroxide concentrations were quantitatively analyzed using the AmplexRed hydrogen peroxide/peroxidase assay kit (Invitrogen).

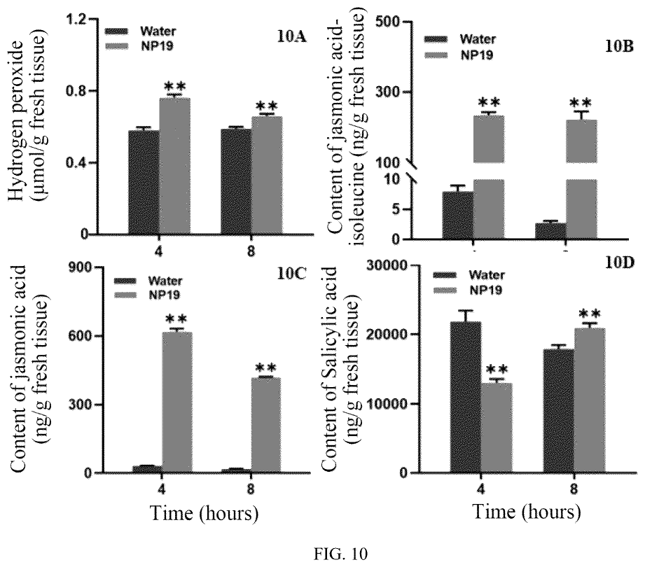

Results: The results of quantitative analysis of hormone concentrations showed that NP19 could significantly induce an increase in the contents of hydrogen peroxide, jasmonic acid, and jasmonic acid-isoleucine, as shown in FIG. 10. Among them, 10A shows the content of hydrogen peroxide, 10B shows the content of jasmonic acid-isoleucine, 10C shows the content of jasmonic acid, and 10D shows the content of salicylic acid.

### Embodiment 6 Induction of Insect Resistance in Tobacco and Rice by the Short Peptide NP19

(1) Induction of Resistance in Tobacco Against Bemisia tabaci

Fifteen Bemisia tabaci adults were placed in the center of each pair of leaves treated with the NP19 short peptide or water. The number of insects on each leaf was recorded at 1, 2, 4, 8, 12, 24 and 48 hours.

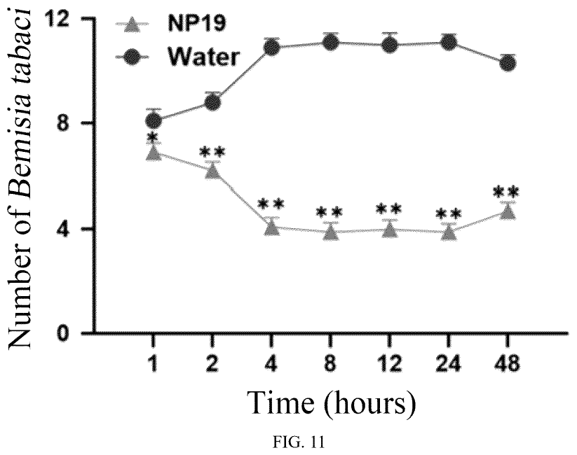

Results: After treating Nicotiana benthamiana leaves with the NP19 short peptide, the number of insects could be significantly reduced, indicating that the NP19 short peptide can induce the resistance of Nicotiana benthamiana against Bemisia tabaci, as shown in FIG. 11.

(2) Induction of Resistance in Rice Against Nilaparvata lugens (Stal)/Laodelphax striatellus (Fallen)

One rice plant with its stem smeared with the short peptide NP19 and another rice plant with its stem smeared with pure water were sealed in a glass cylinder (with a diameter of 8 cm and a height of 8 cm), and 15 fourth-instar nymphs were released. The number of Nilaparvata lugens (Stal)/Laodelphax striatellus (Fallen) nymphs on each plant was counted at 1, 2, 4, 8, 24, and 48 hours after release.

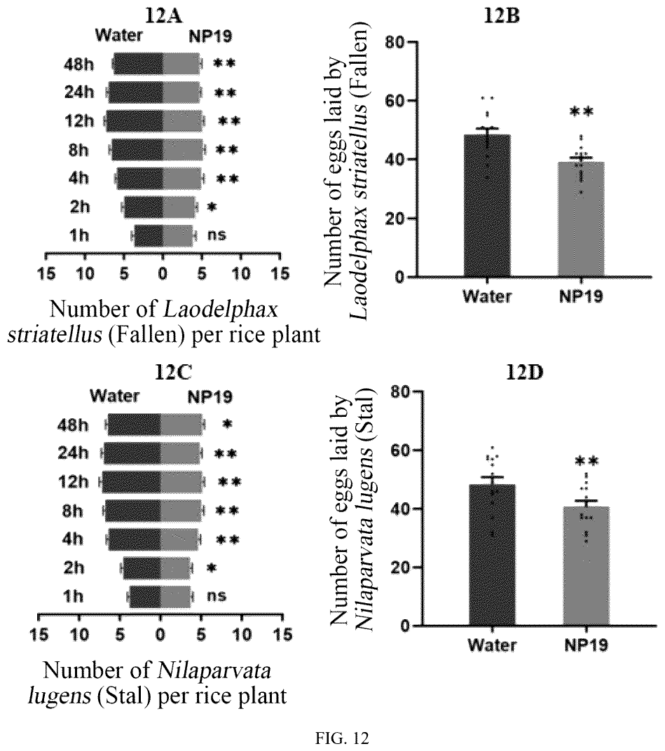

Results: After smearing with the short peptide NP19, the number of Nilaparvata lugens (Stal)/Laodelphax striatellus (Fallen) residing on rice could be significantly reduced, and the egg-laying amount of both Nilaparvata lugens (Stal) and Laodelphax striatellus (Fallen) also decreased significantly, indicating that NP19 could induce the resistance of rice against Nilaparvata lugens (Stal) and Laodelphax striatellus (Fallen), as shown in FIG. 12. Among them, 12A shows the number of Laodelphax striatellus (Fallen) on each rice plant, 12B shows the egg-laying amount of Laodelphax striatellus (Fallen), 12C shows the number of Nilaparvata lugens (Stal) on each rice plant, and 12D shows the egg-laying amount of Nilaparvata lugens (Stal).

(3) Induction of Resistance in Rice Against Chilo suppressalis

One rice plant with its stem smeared with the short peptide NP19 and another rice plant with its stem smeared with pure water were sealed in a glass cylinder (with a diameter of 8 cm and a height of 8 cm). After weighing, five second-instar nymphs were placed into the glass cylinder and attached to the basal stems. After 7 days of feeding, each insect was reweighed to evaluate the weight gain.

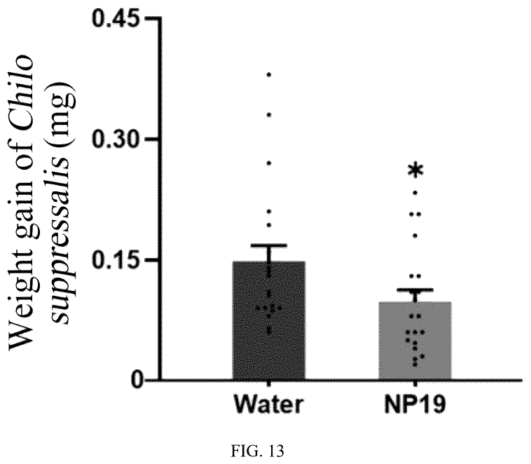

Results: After treating rice with the short peptide NP19, the feeding of Chilo suppressalis could be significantly reduced, indicating that NP19 can induce the resistance of rice against Chilo suppressalis, as shown in FIG. 13.

### Embodiment 7 Induction of Cell Death in Different Plants by the Short Peptide NP19

The short peptide NP19 was used to treat plant leaves. For dicotyledonous plants (cotton, eggplant), the short peptide was adjusted to a concentration of 0.5 m and then infiltrated into the leaves of different species using a needleless syringe. For monocotyledonous plants (rice, corn), micro-wounds were created using quartz sand, and the peptide solution was then infiltrated into the plants.

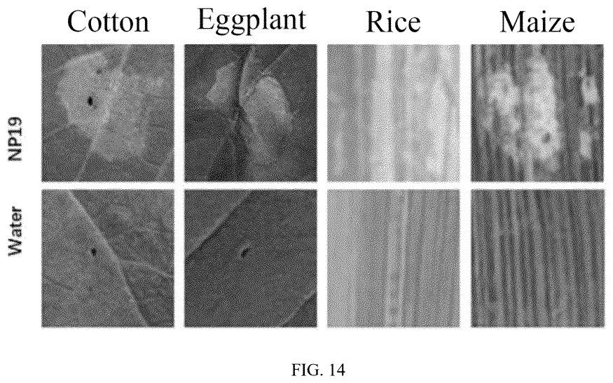

Results: The short peptide NP19 could induce cell death in the leaves of cotton, eggplant, rice, and maize, as shown in FIG. 14.

### Embodiment 8 Induction of Insect Resistance in Cotton by the Short Peptide NP19

Selectivity test: Eight second-instar Helicoverpa armigera larvaes or fifteen female Aphis gossypii adults were introduced into the center of each pair of cotton leaves treated with 0.5 μM NP19 short peptide or water. The number of insects on each leaf was recorded at 1, 2, 4, 8, 12, 24, and 48 hours.

Each pair of cotton leaves treated with 0.5 μM NP19 short peptide or water were placed in a culture dish, and four Aphis gossypii adults were placed on each leaf. After 3 days, the newly born offsprings were counted under a microscope.

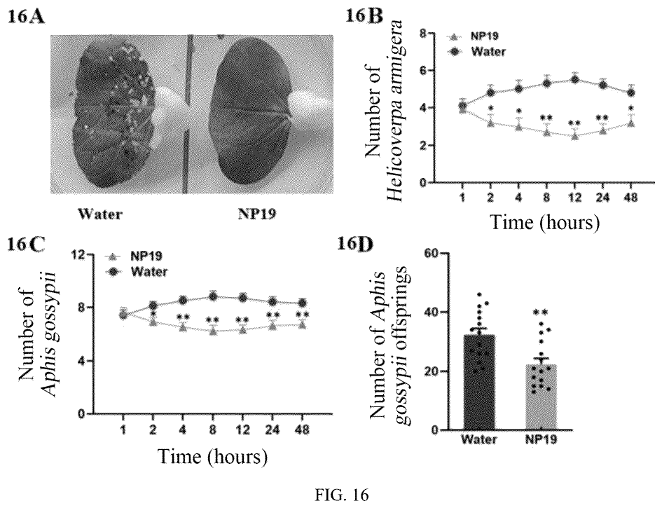

Results: Compared with cotton leaves treated with the short peptide NP19, Helicoverpa armigera larvae or Aphis gossypii tended to feed on water-treated cotton leaves. Compared with cotton leaves treated with the short peptide NP19, the reproductive capacity of Aphis gossypii on the water-treated cotton leaves was significantly stronger. It indicated that NP19 can induce cotton to develop resistance against Helicoverpa armigera or Aphis gossypii, as shown in FIG. 16. Among them, 16A shows the growth picture of Aphis gossypii or Helicoverpa armigera on cotton leaves, 16B shows the statistical curve of the number of Helicoverpa armigera, 16C shows the statistical curve of the number of Aphis gossypii, and 16D shows the statistical curve of the number of Aphis gossypii offsprings.

The present disclosure has been disclosed above with reference to the preferred embodiments, but it is not intended to limit the present disclosure. Any technical solutions obtained by equivalent substitutions or equivalent transformations fall within the protection scope of the present disclosure.

What is claimed is:

1. A short peptide NP19 derived from salivary sheath secretory protein of Nilaparvata lugens (Stal), wherein the amino acid sequence of the short peptide NP19 is as set forth in SEQ ID NO: 3.

2. A nucleotide sequence encoding the short peptide NP19, wherein the nucleotide sequence is as set forth in SEQ ID NO: 4.

3. Application of the short peptide NP19 according to claim 1 or a polypeptide Nlsp5 comprising the short peptide NP19 in the preparation of a plant insect-resistant preparation for controlling Laodelphax striatellus (Fallen), Nilaparvata lugens (Stal), Bemisia tabaci, Helicoverpa armigera, Sogatella furcifera (Horvith) or Chilo suppressalis, wherein the amino acid sequence of the polypeptide Nlsp5 is as set forth in SEQ ID NO: 1.

4. The application according to claim 3, wherein the short peptide NP19 or the polypeptide Nlsp5 is used as an elicitor to induce defense responses or hypersensitive responses in a plant.

5. The application according to claim 3, wherein the short peptide NP19 is sprayed on tobacco or rice as a pesticide or as a main component of a pesticide.


SEQUENCE LISTING (16 sequences)

SEQ ID NO: 1 - protein, 225 aa, synthetic construct
  source 1..225: note = the amino acid sequence of the polypeptide Nlsp5
MASFTSLLSALVLVAGSQGVAVNPMGMADAGLGMVDAGVSAGMGLASTGMDAGKGLANAGMGAASSMSGRMTEEAKKLNGNIQDMLGAQMKGGANILNNMLKMDAAVLTEMGTMISSIAHLTGATSQQMLQILQEVMTKGPLAGAQMLQRLLAYIAQKKIAAGDKIVSKTSSFADGMKQKSFIPLNMLDNMYRTLGSVNDITSALTGTPSLAASKAATALTPPVV

SEQ ID NO: 2 - DNA, 678 bp, synthetic construct
  source 1..678: mol_type = other DNA; note = the nucleotide sequence encoding the polypeptide Nlsp5
atggcttcattcacatctctattatccgctcttgtgttggtcgctggttcacagggagtggctgtgaacccaatgggaatggcggatgcaggactgggaatggtggatgcaggagtttcagcgggaatgggactggcaagtactggaatggatgcaggaaagggcttggcgaatgcaggaatgggtgctgctagttccatgtctggtcgcatgacggaggaagctaaaaaattaaatggaaacattcaggacatgttaggagcgcagatgaagggaggtgcaaacatactgaataatatgttgaagatggacgctgccgttctcactgaaatggggacaatgatcagttctattgcacacttaactggagcaacatcacaacagatgttgcaaatactacaagaagtcatgacgaaaggaccactcgcaggtgctcaaatgttacagaggttattagcatacattgcacaaaagaagatagcagcaggtgataagattgtttccaagaccagcagcttcgctgatggcatgaagcaaaaatcgtttataccattaaacatgttggataatatgtacagaacgcttggatcagttaacgatataacatcagcattgactggcactccttcgcttgccgcatcaaaagcagctacagccctcactccacccgtagtttga

SEQ ID NO: 3 - protein, 19 aa, synthetic construct
  source 1..19: note = The amino acid sequence of the short peptide NP19
KGPLAGAQMLQRLLAYIAQ

SEQ ID NO: 4 - DNA, 57 bp, synthetic construct
  source 1..57: mol_type = other DNA; note = the nucleotide sequence encoding the short peptide NP19
aaaggaccactcgcaggtgctcaaatgttacagaggttattagcatacattgcacaa

SEQ ID NO: 5 - DNA, 20 bp, synthetic construct
  source 1..20: mol_type = other DNA; note = The primers for PCR screening of the strains
gttaggagcgcagatgaagg

SEQ ID NO: 6 - DNA, 20 bp, synthetic construct
  source 1..20: mol_type = other DNA; note = The primers for PCR screening of the strains
acatttgagcacctgcgagt

SEQ ID NO: 7 - DNA, 21 bp, synthetic construct
  source 1..21: mol_type = other DNA; note = the primers for verifying the expression of the Nlsp5 gene
agattgtttccaagaccagca

SEQ ID NO: 8 - DNA, 21 bp, synthetic construct
  source 1..21: mol_type = other DNA; note = the primers for verifying the expression of the Nlsp5 gene
cgttaactgatccaagcgttc

SEQ ID NO: 9 - DNA, 18 bp, synthetic construct
  source 1..18: mol_type = other DNA; note = primer of NbEF1a-QF
agaggccctcagacaaac

SEQ ID NO: 10 - DNA, 18 bp, synthetic construct
  source 1..18: mol_type = other DNA; note = primer of NbEF1a-QR
taggtccaaaggtcacaa

SEQ ID NO: 11 - DNA, 20 bp, synthetic construct
  source 1..20: mol_type = other DNA; note = primer of NbPR3-QF
tggggttattgctggcttag

SEQ ID NO: 12 - DNA, 20 bp, synthetic construct
  source 1..20: mol_type = other DNA; note = primer of NbPR3-QR
gggtcatccaaaaccagaga

SEQ ID NO: 13 - DNA, 22 bp, synthetic construct
  source 1..22: mol_type = other DNA; note = primer of NbPR4-QF
ggccaagattcctgtggtagat

SEQ ID NO: 14 - DNA, 25 bp, synthetic construct
  source 1..25: mol_type = other DNA; note = primer of NbPR4-QR
cactgttgtttgagttcctgttcct

SEQ ID NO: 15 - DNA, 19 bp, synthetic construct
  source 1..19: mol_type = other DNA
tactggaatggatgcagga

SEQ ID NO: 16 - DNA, 20 bp, synthetic construct
  source 1..20: mol_type = other DNA
acatttgagcacctgcgagt